(19) United States
(12) Patent Application Publication
(10) Pub. No.: US 2006/0161196 A1
(43) Pub. Date: Jul. 20, 2006

(54) Methods of and apparatus for use in medical treatment

(76) Inventor: Alan David Widgerow, Gauteng (ZA)

Correspondence Address: CHRISTIE, PARKER & HALE, LLP, PO BOX 7068, PASADENA, CA, 91109-7068, US

(21) Appl. No.: 11/333,840

(22) Filed: Jan. 17, 2006

Related U.S. Application Data
(60) Provisional application No. 60/593,481, filed on Jan. 18, 2005.

(30) Foreign Application Priority Data
Aug. 13, 2005 (ZA) 2005/06174
Oct. 26, 2005 (ZA) 2005/08678

(51) Int. Cl. A61M 29/02; A61F 2/12

(52) U.S. Cl. 606/192; 623/8; 604/97.01

(57) ABSTRACT

Equipment and methods for preparing a person for placement of a subcutaneous prosthesis, e.g., are disclosed. An inflatable expander, such as an expandable bladder, is introduced under an area of skin to be stretched. A tube extends through the skin to a preferably valved fluid flow connector. The person whose skin is to be stretched can periodically introduce desired amounts of fluid, such as saline solution, into the expander by use of a patient controlled delivery machine (“PCD machine”) which is connectible to the fluid flow connector. The PCD machine can be a syringe having an outlet fitting which is matable to the flow connector. Since the person controls the times and amounts of liquid added to the expander, there is a reduction in the time, cost, and discomfort encountered by the person in preparation for placement of a prosthesis under stretched area of skin.

### CROSS-REFERENCE TO RELATED APPLICATIONS

This application claims the foreign priority benefits, under 35 U.S.C. 119(a), of Republic of South Africa applications numbers 2005/06174 filed 16 Aug. 2005 and 2005/08678 filed 26 Oct. 2005. This application also claims, under 35 U.S.C. 119(e), the benefit of U.S. provisional application No. 60//593,481 filed on Jan. 18, 2005.

### FIELD OF THE INVENTION

This invention relates to methods of and apparatus for use in medical treatment.

### BACKGROUND OF THE INVENTION

After certain surgical procedures it is necessary to insert prostheses under the skin. However, there are often difficulties because the skin is very tight and unable to stretch to accommodate the prosthesis. The procedure presently adopted to meet this problem is to insert a hollow flexible expander under the skin and to increase the volume of the expander by introducing a liquid into it and thereby stretching the skin. The procedure continues until the skin is large enough to accommodate the prosthesis there below with the patient remaining comfortable. The prosthesis is then permanently inserted in position. The same procedure may be adopted to repair skin on the head, neck, hands, arms and legs where, although a prosthesis is not used, skin has to be stretched to replace damaged or removed skin. It will be understood that in certain circumstances, other tissue may also have to be stretched/expanded and the term “skin” as used herein is intended to cover such tissue, unless clear from the context.

In a known procedure, the expander is inserted under the skin and a permanently attached flexible tube passes through the skin into the external environment. A sealed receiver unit/valve, which is covered by a membrane, is connected to the distal end of the flexible tube and buried beneath the skin or may be integrated into the expander. The patient now visits a medical practitioner, normally a surgeon, who introduces the liquid into the expander thereby enlarging it to stretch the skin. This the medical practitioner does by using a fine needle attached to a syringe to pierce the skin and valve membrane and to feed liquid into the valve/receiver unit from whence it passes via the flexible tube into the expander. It will be understood that the stretching of the skin must take place in small increments. If the skin is stretched to too great an extent, the patient will suffer discomfort or pain, and in extreme cases the scar tissue may tear. This limits the amount of expansion of the expander during each visit to the medical practitioner. Consequently the treatments required which normally take place on a weekly basis, are numerous, and, for example, in the case of stretching skin after a mastectomy, the procedures may extend over a period of as long as three months. Furthermore, of course, the procedure is expensive and inconvenient requiring the patient to visit the practitioner (usually necessitating travel, waiting in the reception room etc). In addition until the reconstructive surgery takes place, the patient will suffer from abnormal due to the expansion as aforesaid. There will also be increased risk of complications where the expansion takes place over and extended period of time. For all these reasons it is desirable for the expansion period to be decreased which would contribute to patient convenience, decreased costs and improve acceptance of this technique.

### SUMMARY OF THE INVENTION

According to one aspect of the invention there is provided apparatus for use in expanding skin, comprising an inflatable expander that is capable of being introduced under the skin of a patient which is required to be stretched, a flexible tube leading from the expander and being capable of passing through the skin of the patient, and a patient controlled liquid delivery machine (hereinafter called a “PCD machine”) connected the flexible tube or connectable to the interconnector whereby a patient may deliver liquid via the interconnector and the flexible tube to the expander. Preferably an inter-connector member is located at the distal end of the flexible tube and connected to the PCD machine.

Normally a patient using this apparatus will not deliver too much liquid by means of the PCD machine because discomfort and pain will prevent such excessive delivery. Preferably, however, the PCD machine incorporates means for ensuring that only limited amounts of liquid can be dispensed thereby during a specified time period.

Preferably connector means are provided between the interconnector and the dispensing end of the PCD machine, which are easily cleanable so that the patient may maintain the apparatus sterile. A covering is normally provided for the interconnector, so that this is protected when not in use. Similarly, a covering member may be provided for the dispensing end of the PCD machine.

According to another aspect of the invention, there is provided a method of expanding skin, comprising using at the apparatus as set forth above, the method comprising inserting an expander under the skin, passing the flexible tube through the skin and connecting an interconnector thereto, attaching a PCD machine to the interconnector and permitting the patient to use the PCD machine to introduce liquid into the interconnector and thereby delivering this to the expander.

An embodiment of the invention will now be described by way of example, with reference to the accompanying drawings.

### BRIEF DESCRIPTION OF THE DRAWINGS

In the drawings:

FIG. 1 is a perspective view of an expander of the invention, incorporating a trocar,

FIG. 2 is a perspective view of an intermediate connector set,

FIG. 3 is a perspective view of the apparatus of the invention, and

FIG. 4 is a diagrammatic view showing the position of the expander applied to a patient who has had a mastectomy.

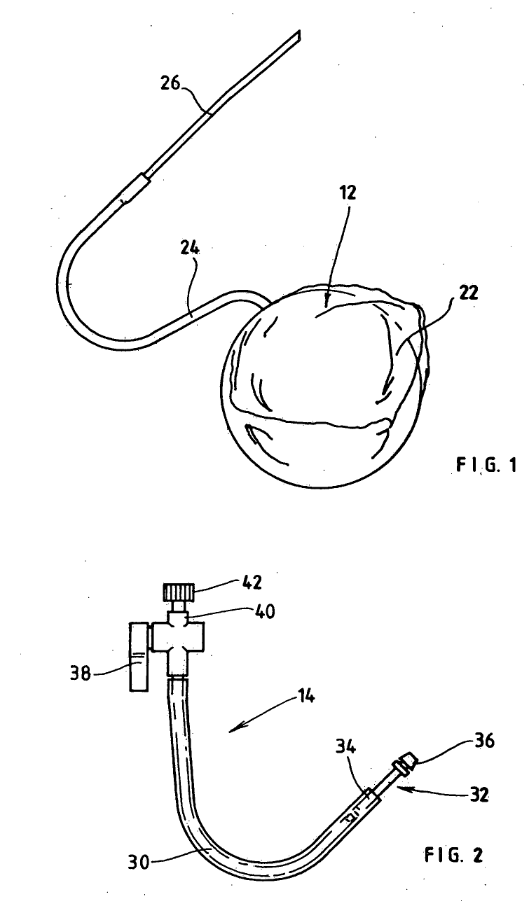

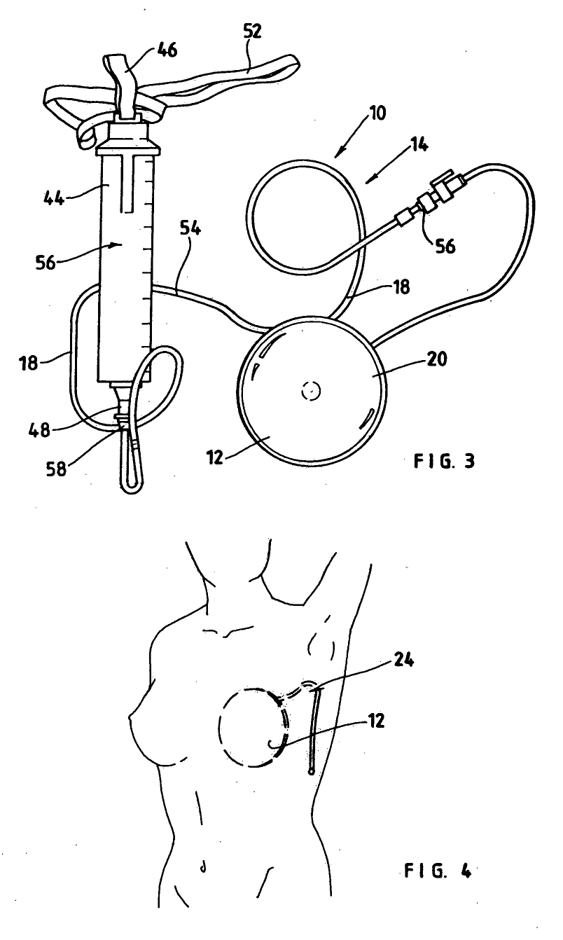

### BRIEF DESCRIPTION OF THE INVENTION

Referring now to the drawings, apparatus 10 of the invention comprises an expander 12, an intermediate connector set 14 and a PCD machine 16 (consisting of a controlled syringe) together with a connector pipe 18.

The expander 12 comprises a hollow plastic member having a reasonably rigid circular base 20 and a collapsible part spherical top part 22 formed of flexible and expandable plastic material. A permanently connected flexible silicone tube 24 is connected to the expander 12, adjacent to the base 20. At its free end, the tube 24 is connected to a trocar 26. When a liquid is introduced into the expander 12, as will be described below, the volume of the expander increases and thus stretches the skin covering the expander 12.

The intermediate connector 14 comprises a flexible silicone tube 30 having at one end a metal union or connector 32. The connector 32 comprises a first spigot 34 that fits into the proximal end of the tube 30 and a second spigot 36 which in use fits into the distal end of the tube 24 when the trocar 26 is removed. The distal end of the tube 30 is attached to an interconnector valve 36 incorporating a one way valve or stopcock 38 which permits fluid flow to the tube 30 but prevents escape of liquid via the tube 30. The interconnector valve 36 has a socket or female Luer adapter 40. A covering cap 42 fits into and covers the adapter 40 to provide covering therefor when not in use.

The PCD machine comprises a cylinder 44 incorporating an operating lever 46 at one end and a male Luer adapter 48 at the other end. The cylinder 44 also incorporates a control mechanism (not shown) which, when the operating lever 46 is depressed, emits a pre-set amount of liquid. Typically, the syringe 16 may be a Freedom (7301) patient controlled analgesia (“PCA”) device supplied by Vygon SA and some details of which are described in U.S. Pat. No. 6,595,981.

However other similar products may be used, preferably with easily refillable components. A female Luer lock obturator (not shown) provides temporary capping for the male Luer adapter 48. A carrier cord 52 is provided to enable the patient to have ready access to the syringe 16 and to have it ready for use in an ambulatory manner.

The connector pipe 18 is of an appropriate length, as will be described below, and consists an elongated silicone tube 54 having at one end a male Luer adapter 56 for engaging in the female Luer adapter 40 of the intermediate connector 14 and at the other end a female Luer adapter, 58, to be engaged by the male Luer adapter 48 on the controlled syringe 16. Again many variations in design are possible.

In use, after a mastectomy, the expander 12, which is in the collapsed condition, is surgically fitted under the skin retropectorally on the mastectomy side in the normal fashion (see FIG. 4). By means of the trocar 26 an opening is made in the skin, preferably below the arm pit, so that the tube 24 is exposed to the external atmosphere. The trocar 26 is now removed by cutting the end part of the tube 24. The second spigot 36 is inserted into the free end of the tube 24. The stopcock 38 is closed, and the cap 42 covers the adapter 40. A small amount of liquid is introduced into the expander by the surgeon. The exposed part of the tube 24 is bound to the patient in a way to protect this item and to cause the patient minimum discomfort.

After a period of about 5 to 14 days after the implant procedure the patient may now commence expansion or sooner in some cases when the surgeon believes applicable. The patient uses the connector pipe 18 to connect the PCD machine to the intermediate connector 14. By depressing the lever (or button) 46, the patient ensures that a preset amount of liquid (normally saline) into the expander 12 via the intermediate connector 14 and the tube 24. The PCD machine prevents excessive introduction of liquid into the expander 12 and usually no more than 5-7 millilitres of saline are introduced during each procedure. However, the procedure can be repeated multiple times a day so that the skin is stretched to a small extent after each procedure. After each stretching operation the skin relaxes, so that during the next stretching operation the skin will be ready to stretch further and pain will be reduced.

The patient may apply topical creams to the skin being stretched to facilitate the process of stretching of the skin and to ease any pain involved. It is desirable that the surgeon or the medical practitioner monitors the process once or twice weekly which may or may not involve visits to the surgeon or medical practitioner. The process is continued until the breast is slightly over expanded which should be determined by the surgeon and is left as such until the next stage is scheduled. The expander is then removed and the normal routine of the breast reconstruction is continued.

We have found that it is relatively easy to instruct the patient how to operate and refill the PCD and how to keep the various parts clean and sterile.

We have found that because the expansion takes place regularly with very small increments, it is possible to complete the fill expansion of the expander in a very short period of time such as typically three weeks. Thus the reconstruction time can be decreased by about two months or more. Further as the visits to the medical practitioner can be reduced significantly the cost of the procedure will also be considerably reduced.

The invention is not limited to the precise details hereinbefore described and illustrated. For example the same procedure may be adopted for any case of tissue expansion e.g. to repair skin on the head, neck, hands, arms and legs where, although a prosthesis is not used, skin has to be stretched to replace damaged or removed skin. The shape of the expander would vary according to the needs of the reconstruction. The expander may vary as will be appreciated by those skilled in this art. The use of procedure on the skin on the head has a benefit in that hair will grow through this skin which usually does not occur where skin is taken from another part of the patients body. The amount of liquid provided on each operation of the PCD machine may vary as desired. Other liquids may be used to enlarge the expander.

Many variations of PCD machines may be used including soft walled reservoirs instead of cylinders, control mechanisms such as buttons detached from the reservoir units, reservoir units that are refillable in a variety of ways. A variety of PCD machines have been used for patient directed pain control.

The interconnector may be dispensed with according to the design of the PCD. Thus for example the flexible tube may incorporate a one way valve to prevent escape of fluid and have a cap for keeping the end sterile. This cap will be removed prior to connection of the tube to the PCD.

In a further modification pressure reading monitors may be attached to the expander tube enabling the monitoring of pressure transmitted within the expander and on to the overlying skin.

Future scenarios are envisaged where teletype monitoring from home is possible, whereby photo or video documentation of the status of the expansion is transmitted to the surgical control centre.

I claim:

1. Apparatus for use in expanding skin, comprising
an inflatable expander that is capable of being introduced under the skin of a patient which is required to be stretched,
a flexible tube leading from the expander and being capable of passing through the skin of the patient, and
a patient controlled liquid delivery machine connected the flexible tube or connectable to the interconnector whereby a patient may deliver liquid via the interconnector and the flexible tube to the expander.

2. Apparatus as claimed in claim 1 further comprising an inter-connector member located at the distal end of the flexible tube and connected to the PCD machine.

3. Apparatus as claimed in claim 1 wherein the patient controlled liquid delivery machine incorporates means for ensuring that only limited amounts of liquid can be dispensed thereby during a specified time period.

4. Apparatus as claimed in claim 1 further comprising connector means between the interconnector and the dispensing end of the patient controlled liquid delivery machine, said connector means being easily cleanable so that the patient may maintain the apparatus sterile.

5. Apparatus as claimed in claim 1 further comprising a covering provided for the interconnector, so that this is protected when not in use.

6. Apparatus as claimed in claim 1 further comprising a covering member for the dispensing end of the patient controlled liquid delivery machine.

7. A method of expanding skin, comprising using at the apparatus as claimed in claim 1, the method comprising
the surgeon inserting the expander under the skin, passing the flexible tube through the skin and connecting an interconnector thereto,
attaching a patient controlled liquid delivery machine to the interconnector and
permitting the patient to use the patient controlled liquid delivery machine to introduce liquid into the interconnector and thereby delivering this to the expander.
